(19) United States
(12) Patent Application Publication
(10) Pub. No.: US 2018/0207420 A1
(43) Pub. Date: Jul. 26, 2018

(54) Method and apparatus for treating skin

(71) Applicants: ÅBO AKADEMI, Turku (FI); TAMPEREEN TEKNILLINEN YLIOPISTO, Tampere (FI); TEKNOLOGIAN TUTKIMUSKESKUS VTT OY, Espoo (FI)

(72) Inventors: Saara Tuurala, Espoo (FI); Anu Vaari, Espoo (FI); Mikael Bergelin, Turku (FI); Jan-Erik Eriksson, Turku (FI); Atte Kekonen, Tampere (FI); Heimo Ylänen, Tampere (FI)

(21) Appl. No.: 15/106,332

(22) PCT Filed: Dec. 19, 2014
(86) PCT No.: PCT/FI2014/051032

§ 371 Date: Jun. 20, 2016

(30) Foreign Application Priority Data
Dec. 20, 2013 (FI) 20136307

(51) Int. Cl. A61N 1/30; A61M 37/00; A61N 1/04

(52) U.S. Cl. CPC A61N 1/303; A61M 37/00; A61N 1/044; H01M 8/16; A61M 2205/8206; A61M 2205/8268; A61M 2037/0007

(57) ABSTRACT

A method and a device for treating at least a part of an individual's skin having a natural first polarization state. The method comprises the steps of applying at least two electrodes on the individual's skin at a distance from each other, and supplying a direct current to said electrodes to form an electrical potential difference between said electrodes on said part of the skin in order to bringing said part of the skin into a second polarization state, which is different from the first. The part of the skin is then suitable subjected to further treatment for example using cosmetics or pharmaceuticals. The invention can be utilized in the field of cosmetics and for therapy, and it can be used e.g. for accelerating skin care processes.

### TECHNICAL FIELD

The present invention relates to the treatment of an individual's skin. In particular the present invention concerns a method of treating at least a part of an individual's skin, wherein at least two electrodes are placed on the individual's skin at a distance from each other, and a direct current is supplied to the electrodes to form an electrical potential difference between the electrodes on the skin.

The present invention also concerns a method of enhancing delivery of active compounds onto the skin of an individual as well as a device for delivering an active cosmetic or medical agent to an individual's skin.

### BACKGROUND ART

There is an increasing business potential for novel concepts in the high-volume consumer markets for cosmetics products. One of the emerging and steadily growing market areas within the homecare cosmetics sector is galvanic skin treatment. “Galvanic skin treatment” comprises increasing penetration of cosmetic agent (water and or oil based) into the skin.

Since the early twentieth century it has been possible to treat skin with water soluble ions, the water soluble ions being delivered to an individual's skin by iontophoresis, a technique of introducing ionic medicinal compounds into the body through the skin by applying a local electric current. Ionic medicinal compounds are, by definition, charged and thus may enter the body through the skin when the skin has an electrical charge.

Iontophoresis has been used for transdermal or intradermal delivery of substance(s) into the skin.

GB Patent Specification No. 410,009 (1934) describes an iontophoretic device which overcame one of the disadvantages of such early devices known to the art at that time, namely the requirement of a special low tension (low voltage) source of current which meant that the patient needed to be immobilized near such source. The device of the GB Specification was made by forming a galvanic cell from the electrodes and the material containing the medicament or drug to be delivered transdermally. The galvanic cell produced the current necessary for iontophoretically delivering the medicament. This portable device thus permitted iontophoretic drug delivery with substantially less interference with the patient's daily activities.

WO 91/16943 discloses an electrically powered iontophoretic delivery device having a selectively permeable separator membrane positioned between an agent reservoir and an electrode in the device. It also discloses a method which reduces the electrical power requirements of the iontophoretic delivery device to a twenty volt battery or bank of batteries.

US 2003/0135150 discloses an iontophoresis device suitable for effective use of a drug supported on a drug support.

Various kits for controlled iontophoretic delivery of oxidizing agents into the skin are also disclosed in U.S. Pat. Nos. 7,340,297, 7,820,320 and 7,979,117. Thus, e.g., U.S. Pat. No. 7,979,117 discloses a device and method for controlled delivery of active substance into the skin. The device comprises electrodes for electrical coupling to the skin and a separator comprising a porous non-electrochemical cell used is a flexible thin layer.

Further art relating to iontophoresis is disclosed in US 2009221985, WO 0220085 WO 0023144, US 2009270788 and EP 0 616 818.

Applying iontophoretic techniques, the direct current is supplied at relatively high current density in the order of mA/cm², i.e. at a current density that causes the individual to whom the current is applied to be aware of the current and to even suffer discomfort, and at a relatively high potential of up to 30 V.

Iontophoresis may also give rise to a non-specific response (also known as galvanic response, current-induced response, method-induced response). Thus, iontophoresis may introduce a blood flow effect that is not a result of the drug under study. The mechanisms responsible are not completely understood, and the effects are confounding and unpredictable. The cosmetics market is a huge business area and cosmetic brands are always seeking for more efficient ways to benefit from their products. At present a majority of the total volume of galvanic treatments is given to consumers by trained skin care professionals using special equipment. This makes the treatments labour-intensive, time-consuming and expensive. The draw-backs of the iontophoresis makes devices built upon that technology less attractive.

### SUMMARY OF INVENTION

### Technical Problem

In order to make the galvanic treatment widely accessible and inexpensive for the consumers, there is a great need to find new ways to accelerate skin care processes and to be able to do the treatments independently at home, using purpose-made disposable skin care products.

It is therefore an aim of the present invention to overcome at least some of the disadvantages associated with the prior art and to provide a method for treating at least a part of an individual's skin.

It is a further aim of the present invention to provide a method of delivering an active medical agent to an individual's skin.

It is a also aim of the invention to provide a device for delivering an active cosmetic or medical agent to an individual's skin

It is a particular aim of the invention to provide technology which avoids the drawbacks of present galvanic treatments, in particular iontophoresis.

### Solution to Problem

The present method is based on the idea of influencing the surface charge of the outermost layer of the skin, the epidermis only, primarily to change its hydrophilicity. In connection with the present invention it has been found that such a treatment allows for improved wetting of the epidermis.

The aimed effect is achieved by treating at least a part of an individual's skin with low current and voltage so as to change the polarisation state of the treated part of the skin and to contact the treated part, while it is in changed polarisation state, with a cosmetically or therapeutically active compound.

The treatment can be carried out by applying a current to at least one pair of electrodes placed directly on the skin. The electrodes, typically in the form of flexible dermal patches, are spaced apart such as to create a polarisation difference between them.

The invention also relates to a method of delivering an active cosmetic or medical agent to an individual's skin comprising the steps of: altering the polarisation state of the individual's skin, especially a predetermined layer thereof, in particular the epidermal layer, by supplying for a sufficient period of time a direct current to two electrodes positioned on the skin at a distance from each other; and supplying to the individual's skin an active cosmetic or medical agent, either simultaneously with or subsequent to the supply of the current to the skin.

Further, the invention relates to a device in the form of a dermal patch for delivering of an active cosmetic or medical agent to an individual's skin. The device comprises a delivery unit formed by at least one pair of electrodes, including a positive electrode and a negative electrode, both to be contacted with the individual's skin at a distance from each other, such that an electrical current can be generated in the skin to form a potential difference between said electrodes. The device further comprises an electrical power source for providing a current and voltage to the electrodes. More specifically, the methods according to the present invention are characterised by what is stated in claim 1.

The device of the present invention is characterised by what is stated in claim 19.

### Advantageous Effects of Invention

Considerable advantages are provided by the present invention. The treatment with low electric current and voltage avoids the discomfort and drawbacks of traditional galvanic methods while improving wetting and enhancing the natural migration of water and water-soluble components into the epidermis. In particular, the voltage needed for adjusting the polarization state of the skin is much lower than that one applied in iontophoresis, and accordingly non-specific responses are less frequent if not completely inexistent.

The present invention allows for both intradermal and transdermal application of cosmetic am medical active compounds. It is particularly useful for administering skin-moisturizing or anti ageing components on discrete areas of the skin, such as in areas where the skin is particularly thin. An example is facial skin.

The device can be implemented in the form of disposable patches formed by printing technology and connected to a power source which also is formed by printing to give a light-weight, inexpensive, skin polarization instrument. The device is environmentally friendly containing no harmful components, such as heavy metals or toxic chemicals. Since the major parts (electrodes and power source) of the instrument can be produced by printing (roll-to-roll), the price of the product will be reasonable.

Other features and advantages will become apparent from the following description.

### BRIEF DESCRIPTION OF DRAWINGS

Next embodiments will be examined more closely with the aid of a detailed description and with reference to the attached drawings, in which:

FIGS. 1a to 1d show a one biofuel cell device for delivery of an active agent in charged form to an individual's skin.

FIG. 1a shows a sideview and FIG. 1b the top view of one embodiment, whereas FIGS. 1c and 1d show top views of slightly modified embodiments; and

FIGS. 2a to 2c show a two biofuel cell device for delivery of an active agent in charged form to an individual's skin.

FIG. 2a shows a sideview and FIG. 2b section A-A of one embodiment, whereas FIG. 2c shows a top view of an alternative embodiment.

FIG. 3 depicts graphically the change in decrease in impedance as a function of time for two impedance measurements (cf. the test of mechanism discussed in Example 4).

FIG. 4 depicts graphically the change in skin impedance and skin potential as a function of time (cf. test of mechanism in Example 5).

FIG. 5 is a bar chart showing change in moisture of skin depending on time duration after end of treatment, illustrating the lasting effect of the present treatment as well as correlation between measured impedance values and Corneometer™ values.

### DESCRIPTION OF EMBODIMENTS

Various embodiments described herein provide a method of treating at least a part of an individual's skin, which part of the skin has a natural, in the following also referred to as a natural “first” polarisation state. As discussed above, the method comprises the steps of applying at least two electrodes on the individual's skin at a distance from each other; and supplying a direct current to said electrodes to form an electrical potential difference between said electrodes on said part of the skin in order to bring said part of the skin into a second polarisation state, which is different from the first.

Optionally the part of the skin so treated is subjected to further treatment, such as the application of a cosmetically or pharmaceutically active ingredient, such as a moisturizing components or anti-ageing cream. Skin treatment with an active ingredient can also be carried out simultaneously with the application of the electrodes to the skin.

The creation of an electrical potential difference between at least two electrodes on skin, which polarises the skin, will have the effect that the hydrophilicity of the skin is changed allowing improved wetting and enhancing the natural migration of water and water soluble components into the skin.

The electrical potential difference formed between the electrodes can be described as small, in comparison to those required in iontophoretic techniques. In one embodiment the electrical potential difference is smaller than 1.4 V, especially smaller than 1.0 V, in particular smaller than 0.9 V, preferably about 0.1 to 0.8 V, for example about 0.5 to 0.75 V. In another embodiment, the electrical potential difference is about 1.0 V to 1.75 V, in particular about 1.2 to 1.6 V.

In an embodiment of the present invention the direct current is supplied at a current density below sensation threshold, in particular at about 0.1 to 10 μA/cm², preferably about 0.25 to 5 μA/cm².

In another embodiment the electrical potential difference and the current density are selected such that the skin will exhibit the second polarisation state for 0.1 to 300 s, in particular 1 to 180 s after the end of the application of a voltage on the skin. Typically, the device described herein is placed and kept on the skin for about 1 to 600 s before removing the device.

It is also possible to use a device which has an electric switch, in which case the voltage can be cut off by removing the device.

The area (mm²) of the part of the individual's skin that is polarised in the method varies. In one embodiment said part of the individual's skin has an area of at least 1 mm², preferably at least about 5 mm². Although no absolute upper limit can be given, it would seem that areas of the same size as the patient's palm can still be readily treated.

In a further embodiment a predetermined layer of the skin, in particular the epidermal layer, is polarised. Facial skin is a particularly interesting object for the treatment, but the technology can be carried out for any part of the human skin, preferably save for the genital area, as far as cosmetic treatment is concerned.

The polarization effect will penetrate the skin to a depth of approximately 10 nm to 5000 μm, typically about 100 nm to 1000 μm. Preferably it will reach through the top layer of the skin, the Stratum corneum, and penetrate into the epidermis, and optionally through the epidermis to the dermis. As known, the corneum has a thickness from approximately ten to several hundred micrometres, depending on the region of the body.

In a further embodiment the depth into the skin to which the active agent penetrates, is further adjusted
by selecting one or more of the following: appropriate properties of the formulation, such as its pH; viscosity; conductivity; adhesiveness; concentration of the buffer; concentration of the electrolyte and the concentration of the agent in the composition;
by selecting an appropriate period of time that the treatment process is allowed to proceed;
by adjusting the magnitude or the voltage or the electric current; or
by a combination of two or more of the afore-mentioned.

The electrodes are applied on the individual, and in particular they can be applied by the individual himself As explained below in more detail, the electrodes are preferably connected to a wearable power source for example of a kind which can be activated by moisturizing.

According to one embodiment at least one of the electrodes is applied directly on the part of the individual's skin which is to be treated. From the moment that at least one electrode has been applied on the part of the individual's skin and current applied to the electrodes, the part of the individual's skin (and the region adjacent to it) is polarised and exhibits a second polarisation state. The second polarisation state is maintained after removal of the electrode or electrodes from the individual's skin for up to 300 s as detailed above.

As a practical simplification the skin areas under each electrode can be viewed as electronic extensions of the conductors. In reality the situation is significantly more complex, with the electrodes of the patch being in both electronic and ionic contact with the skin area under each electrode. Of these the ionic conductivity is significantly larger than the electronic conductivity. Therefore the mechanism may be viewed as a system of three serially connected electrochemical double layer capacitors. Capacitors 1 and 2 consists of a patch electrode, the skin area under said electrode acting as the second capacitor electrode, and a volume of emulsion or gel between said capacitor electrodes acting as the electrolyte. The third capacitor is formed from the skin areas in capacitor one and two, where intercellular fluid in the interface between the dermis and epidermis acts as electrolyte.

Polarisation of a part of the skin enhances the migration of water and water soluble components into that part of the skin. The active component can be introduced at any suitable point of time from the beginning of the polarisation process up to the point, when the difference in polarisation state between the selected part of the skin and the surrounding parts of the skin has disappeared.

Thus, in one embodiment the active component is adhered to the skin-side of the electrode to be placed upon the skin, so that migration of the active component will take place when the electrode is placed on the skin and electric current is applied to the pair of electrodes. In another embodiment the step of subjecting said part of the skin to further treatment comprises applying an active agent on said part of the skin while it is in said second polarisation state.

The active agent can have medical or non-medical properties. In one embodiment a non-medical agent, preferably a non-medical agent selected from cosmetic agents, in particular rejuvenating agents or moisturizing agents, is applied on the skin while it is in the second polarisation state. Specific examples comprise skin moisturizers, including ingredients, such as naturally occurring skin lipids and sterols, artificial or natural oils, humectants, emollients, and lubricants.

Anti-ageing creams containing as ingredients retinol, for example in the form of retinyl palmitate, Epidermal growth factor, alpha hydroxyl acids, beta hydroxyl acids and other chemical peels peptides, coenzyme Q10, argireline, anti-oxidants, sunscreens and vitamin B5 and vitamin C.

Various therapeutically useful compounds can also be introduced intradermally and transdermally using the present technology. As well-known in the art, transdermal application is preferred for drug delivery which needs to be unaffected by food or gastrointestinal problems; avoidance of first-pass metabolism in the patient's liver; and diminished likelihood of hepatic induction.

The agent can be supplied in various forms and formulations. In one embodiment the agent is supplied as a topical formulation such as an ointment, emulsion, lotion, solution or the like. In one embodiment the current density and the voltage are selected to allow for transdermal administration of the agent into the epidermal layer of the skin.

In a further embodiment said part of said skin is healthy, non-wounded skin, and the treatment is primarily intended for cosmetic purposes.

A selection of current sources is available for use. In one embodiment a wearable current source is used. In a further embodiment the wearable current source is a battery with stored electrical energy or the current source is a fuel cell, preferably an enzymatic biofuel cell. A semi-enzymatic biofuel cell is also used in one embodiment. In such an embodiment the fuel cell comprises a biocathode comprising an enzyme e.g. laccase and a non biological anode, or optionally the fuel cell comprises a non biological cathode and a bioanode comprising an enzyme e.g. glucose oxidase.

By means of the present technology, changes in polarisation levels of the skin are achieved.

The low currents used are of a magnitude typical of maintaining the charge separation in a double layer capacitor. The current profile is also typical of a charging behavior with a short term higher current which gradually decreases as the “capacitor gets fully charged”. In electrophoretically forced migration the currents are typically three orders of magnitude larger, and the current profile is plateau-shaped (as the current flow is linearly proportional to the amount of ions moved).

As pointed out above, electrophoretically enhanced migration requires significantly larger potential differences, from at a minimum 1.5 to 30 V. The process is also slow at low potentials (albeit depending of ion size and physical properties of the medium in which they move).

Despite the minute current flow during a short treatment period, the effect is very clear. This has also been verified using a Corneometer (commercial instrument to measure skin hydration). Measurements show a 12-15% higher moisturization level than in comparison tests without polarization, and this effect can still be seen several hours after treatment.

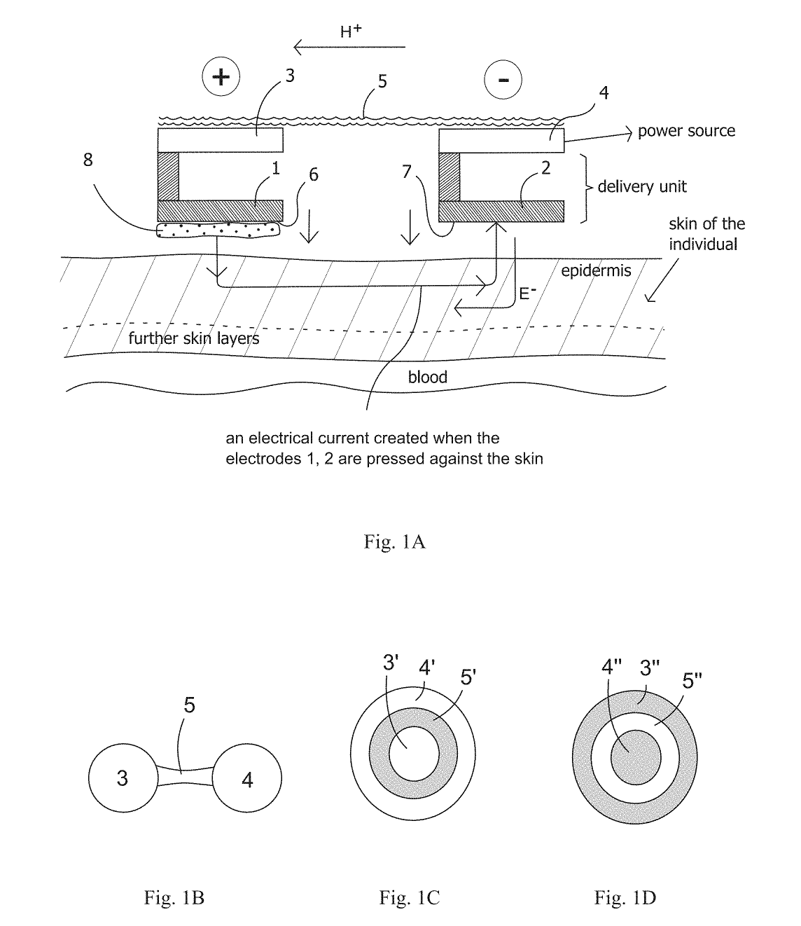

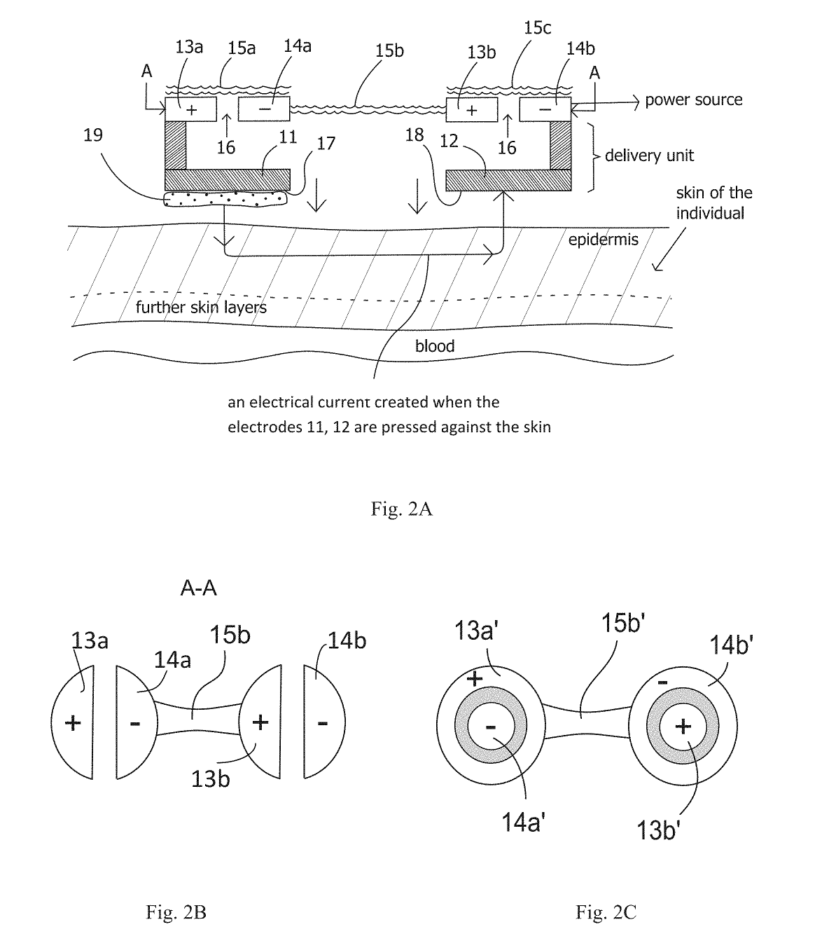

The embodiments shown in the attached figures relate to a device for the delivery of an active agent to an individual's skin. The devices comprise (FIGS. 1a to 1d) an anode 4 and a cathode 3 and an electrically conductive component 5, such as a salt bridge between said anode and cathode and, in the case of more than one biofuel cell (cf FIGS. 2a to 2c), a printed lead 15, between a terminal anode 14a of a first cell 13a, 14a, 15a and a terminal cathode 13b of a final cell 13b , 14b, 15b, the cells connected in series, the delivery unit and the power source being included in the patch.

The embodiment of FIG. 1 discloses a device in the form of a patch for delivering of an active cosmetic or medical agent in charged form, such as in ion form or in polar form, or neutral form if water soluble to an individual's skin.

The device comprises a delivery unit comprising a pair of electrodes 1, 2 including a positive electrode 1 and a negative electrode 2, both to be contacted with the individual's skin, wherein an electrical current in the skin can be generated to form a potential difference between said electrodes 1, 2. The device also comprises an electrical power source with a biofuel cell (not shown) which can be activated with at least one enzyme for providing a current and voltage to said electrodes 1, 2.

In another embodiment one of the electrodes is capable of being contacted with the skin via a layer of conductive substances.

In a further embodiment the conductive substance is an active medical, or cosmetic agent (8, 19).

In a preferred embodiment, which is particularly suitable for producing a device source by printing, cathode 3 comprises a conductive layer containing an enzyme, such as a peroxidase or oxidase, preferably in combination with and an electron transfer mediator. The cathode catalyzes reduction of ambient oxygen to water by uptake of electrons. The device has an anode 4 comprising a conductive layer containing an enzyme, capable of oxidising or dehydrogenating a carbohydrate, preferably in combination with an electron transfer mediator, the conductive layers of both anode and cathode being dry layers. Finally, there is a fuel layer comprising essentially dry carbohydrate, wherein said anode catalyses oxidation of the carbohydrate, thereby releasing electrons. Alternatively, sugar may be dissolved in a solution, which is then added when the electrochemical cell is activated.

Generally, in an embodiment, an active agent is administered to the individual by placing a formulation comprising the active agent in contact with the surface of the positive electrode or the surface of the negative electrode, or a combination of both, before pressing the electrode or electrodes against the individual's skin.

In a preferred embodiment a positively charged active agent, such as a cation, a positively charged liposome or the like including the active agent 8 is administered to the individual by placing a formulation comprising the active agent in contact with the surface 6 of the positive electrode 1 before pressing this electrode against the individual's skin. FIG. 1b shows a top view of the cathode 3 and anode 4 coupled together by an ionic conductor, in the instant embodiment the salt bridge 5.

FIGS. 1c and 1d show top views of alternative embodiments, wherein the electrodes are concentrically arranged, either the cathode 3′ inside the surrounding anode 4′ which is coupled to the cathode by a layer of a salt bridge 5′ in annular configuration (in section).

In FIG. 1d the anode 4″ is arranged on the inside of a salt bridge layer 5″ and the cathode layer 3″. The arrangements of FIGS. 1c and 1d will allow for compact construction of the device.

The embodiment of FIG. 2 is similar to that of FIG. 1 except that there is shown a two cell configuration. Two electrodes 13a; 14a and 13b; 14b, respectively, are coupled in pairs by salt bridges 15a and 15b. The anode 14a of the first pair is coupled with the cathode of the second pair 13b with a lead connector which can be printed.

The two-cell configuration will allow for a placing of the electrodes at a greater distance from each other. Just as in the embodiment of FIG. 1, the anode 14b is connected to a power source.

Just as in FIG. 1, an electrical current is created through the skin when the electrodes 11 and 12 are pressed against the skin.

Further embodiments describe delivering an active medical agent to an individual's skin. One particular embodiment describes a method for delivering an active medical agent to an individual's skin comprising the steps of:

altering the polarisation state of the individual's skin, especially a predetermined layer thereof, in particular the epidermal layer, by supplying for a sufficient period of time a direct current to two electrodes positioned on the skin at a distance from each other; and supplying to the individual's skin an active medical agent, either simultaneously with or subsequent to the supply of the current to the skin.

The application of a medical agent is carried out as discussed above, and the embodiment suitable for application of cosmetic compounds can be applied to therapeutically active compounds as well.

For medical treatment, transdermal drug delivery is of particular interest. As explained above, the depth into the skin to which the active agent penetrates, is further adjusted by selecting properties of the formulation, such as its pH; viscosity; conductivity; adhesiveness; concentration of the buffer, and concentration of the electrolyte and the concentration of the agent in the composition; and by selecting an appropriate period of time that the treatment process is allowed to proceed.

Generally, administration of an active medical agent into the epidermis requires electrical energy at the same level as discussed above: current density of 0.25 to 5 μA cm², and a voltage which is 0.5 to 0.75 V, for intradermal application, or about 1.0 V to 1.75 V, in particular about 1.2 to 1.6 V, in particular for transdermal application.

In an embodiment a positively charged active agent, such as a cation, a positively charged liposome or the like including the active agent is administered to the individual by placing a formulation comprising the a active agent in contact with the surface 6 of the positive electrode 1 before pressing this electrode against the individual's skin.

The power source can be an enzymatic electric cell structure.

In a desired embodiment the enzyme in the cathode of the power source is selected from but not limited to the group of laccases (EC 1.10. 3.2), catechol oxidases (EC 1.10. 3.1), tyrosinases (EC 1.14. 18.1), bilirubin oxidases (EC 1.3. 3.5), peroxidase (EC 1.11. 1.7), manganase peroxidase (EC 1.11. 1.13), lignin peroxidase (EC 1.11. 1.14), cytochrome-c oxidase (1.9.3.1), L-ascorbate oxidase (1.10.3.3) and ceruloplasmin (1.16.3.1).

In a particular embodiment the electron transfer mediator in the cathode of the power source is selected from the group consisting of but not limited to ABTS [2,2′-azino-bis(3-ethylbenzothiazoline-6-sulfonic acid)], methylsyringate [methyl 3,5-dimethoxy-4-hydroxybenzoate] and other methoxy and dimethoxy phenols, and ferrocenecarboxyaldehyde and other ferrocene derivates, and mixtures thereof

In a yet further embodiment the enzyme in the anode of the power source is selected from but not limited to the group of oxidoreductases (EC 1.), including dehydrogenases with NAD+, NADH+, NADP+ or NADPH+ as electron acceptors (EC 1.1.1), e.g. glucose dehydrogenases (1.1.1.47), oxidases with oxygen as electron acceptor (EC 1.1.3) e.g. glucose oxidases (EC 1.1.3.4) and quinoprotein dehydrogenases (EC 1.1.5) e.g. quinoprotein glucose dehydrogenases (EC 1.1.5.2), preferably, the enzyme is selected from quinoprotein glucose dehydrogenase (EC 1.1.5.2) from Gluconobacter oxydans, Gluconobacter suboxydans or Acinetobacter calcoaceticus or glucose oxidase (EC 1.1.3.4) from Aspergillus niger or glucose dehydrogenase (1.1.1.47) from Pseudomonas sp. or from Thermoplasma acidophilum.

In a still further embodiment the electron transfer mediator in the anode of the power source is selected from but not limited to the group consisting of TMPD (N,N,N′,N′-tetramethyl-p-phenylenediamine), tetracyanoquinodimethane (TCNQ), phenazine methosulphate (PMS), hydroquinone, nickelocene and dimethylferrocene, ferrocene, butyl ferrocene, ferrocene acetic acid, hydroxymethylferrocene, ferrocene dicarboxylic acid, ferrocenecarboxyaldehyde and other ferrocene derivates, and mixtures thereof.

Summarizing the features of one particularly preferred embodiment: a potential difference (typically 0.3-1.5 V) to be generated to the skin between two skin contacts is achieved with enzymatic layers: bioanode and biocathode. The application enhances penetration of ions into the skin of both water and or oil based cosmetic. The instrument is totally manufactured by using (but not limited to) printing methods.

### EXAMPLES

### Example 1

### An Instrument Having a Two Electrode Configuration (cf. FIG. 2) was Constructed as Follows

The substrate is a teabag material (Delfortgroup, 24 000 U) laminated with a thin plastic (37.5 μm thick). A bioanode ink (Glucose Oxidase enzyme and Ferrocene methanol mediator mixed into a carbon ink DuPont 7105) is printed on the laminated side of the substrate. The bioacathode ink (Laccase enzyme and ABTS mediator mixed into a carbon ink DuPont 7105) is printed on the laminated side of the substrate, so that a wanted distance is obtained related to the anode ink layer.

Both the anode and cathode inks are cut with a laser (round cut, r=0.5 mm). The distance of the cuts is 10 mm.

On the non-laminated side of the substrate carbon ink (DuPont 7105) is printed on the same “line” as the enzymatic inks. As the ink reaches the through cuts, they form an electrical contact with the enzymatic layers.

The bioanode is connected to the biocathode with teabag material (Delfortgroup, 24 000 U) that is suitable for use as a salt bridge. The material is attached to the printed layer with an adhesive glue.

An adhesive glue (supplied by Kiilto, Tampere, Finland) is printed on the edges of the carbon ink.

### Example 2

### A Double Cell Structure

The substrate is a teabag material (Delfortgroup, 24 000 U) laminated with a thin plastic (37.5 μm thick).

Bioanode ink (Glucose Oxidase enzyme and Ferrocene methanol mediator mixed into a carbon ink DuPont 7105) is printed as two lines on the laminated side of the substrate.

Bioacathode ink (Laccase enzyme and ABTS mediator mixed into a carbon ink DuPont 7105) is printed as two lines on the laminated side of the substrate, so that a wanted distance is obtained related to the anode ink layer.

Outer ink layers are cut with a laser (round cut, r=0.5 mm). The distance of the cuts is 10 mm. On the non-laminated side of the substrate carbon ink (DuPont 7105) is printed on the same “line” as two enzymatic ink layers. As the ink reaches the through cuts, they form an electrical contact with the enzymatic layers.

In single cell, the bioanode is connected to the biocathode with teabag material (Delfortgroup, 24 000 U) that is suitable for use as a salt bridge. The material is attached to the printed layer with an adhesive glue.

Two single cells are connected to each other with a carbon ink.

An adhesive glue (supplied by Kiilto, Tampere, Finland) is printed on the edges of the carbon ink (described on point 5).

### Example 3

### Tests of the Mechanism, Part I

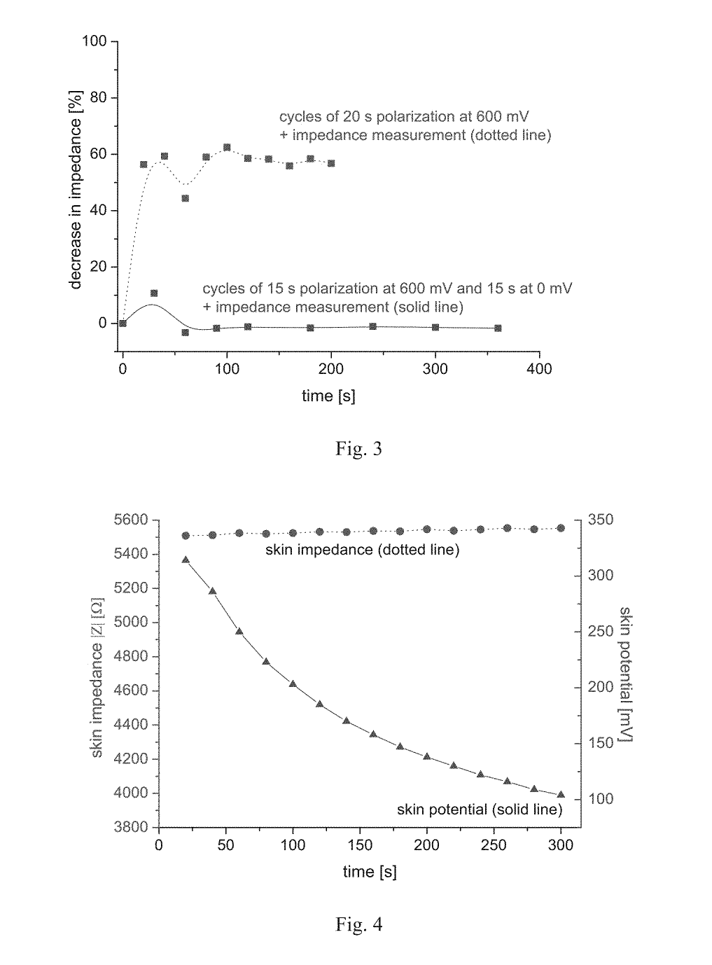

The mechanism is not electrophoretic and the effect is related with maintaining a charged state of the skin as shown in the attached drawing (FIG. 3).

As will appear, the mechanism is not electrophoretic (=iontophoresis). If the electrophoretic effect were responsible for the effect, the two curves below would be virtually the same as the skin's polarization time (=active transfer time for the ions) is practically similar.

If the charged state is not maintained (applying 0 V over the electrodes which discharges the outermost surface of the skin) the result is that virtually no enhanced moisturizing effect can be seen.

### Example 4

### Tests of the Mechanism, Part II

Tissue/skin charging (as generally known) effects can be ruled out as shown in FIG. 4 of the attached drawings.

The figure shows the results of test of the skin impedance change after a 2 min stimulation at 600 mV. As will appear, there is no change in obtained impedance level during 5 min relaxation (decreasing polarization by a factor of 3). Because the impedance does not change as the skin surface discharges, the measured effect is not only related with the charged state of the skin.

### Example 5

### Vertification of Lasting Effect and Correlation Between Measured Impedance Values and Corneometer™ Values

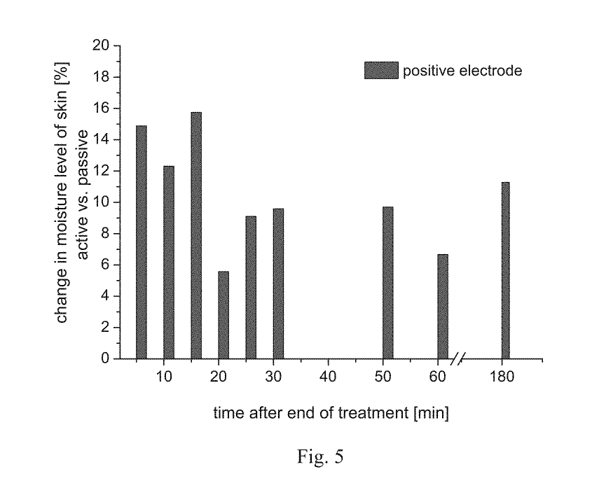

As can be seen from FIG. 5, the effect of the present treatment is lasting.

Skin was moisturized 1) passively and 2) actively under positive and negative electrode on two spots each. Active activation was 10 min at 600 mV. Skin moisture level was measured with Corneometer™ at each point (3 repetitions/spot) for a total of 180 minutes after 10 min moisturizing treatment. Difference of active and passive case for each time point was calculated as percentage difference.

Corneometer™ data clearly supports impedance measurement data and shows that skin polarization has a lasting beneficial effect. The main difference appears to be under the positive electrode with the used skin treatment substance.

### INDUSTRIAL APPLICABILITY

The present invention can be utilized e.g. in the field of cosmetics and for applying pharmaceuticals. In particular, the present technology can be used for accelerating skin care processes and provides for treatments which can be carried out independently at home, for example using purpose-made disposable skin care products. Naturally, the same technology can be used by trained healthcare personnel and carried out at hospitals and institutes.

### REFERENCE SIGNS LIST

1, 11 positive electrode
2, 12 negative electrode
3, 3′, 13, cathode
13a, 13b, cathode
13a′, 13b′ cathode
4, 4′, 14, anode
14a, 14b, anode
14a′, 14b′ anode
5, 5′, salt bridge
15a-15c, salt bridge
15b′ salt bridge
6, 17 surface of the positive electrode
7, 18 surface of the negative electrode
8, 19 formulation of the active substance space between cathode and anode

### CITATION LIST

GB 410,009
WO 91/16943
US 2003/0135150
U.S. Pat. No. 7,340,297
U.S. Pat. No. 7,820,320
U.S. Pat. No. 7,979,117
US 2009221985
WO 0220085
WO 0023144
US 2009270788
EP 0 616 818

1. A method of treating at least a part of an individual's skin, said part of the skin having a natural first polarization state, said method comprising the steps of:
applying at least two electrodes on the individual's skin at a distance from each other, and
supplying a direct current to said electrodes to form an electrical potential difference between said electrodes on said part of the skin in order to bringing said part of the skin into a second polarization state, which is different from the first,
wherein polarizing of the skin and by polarization treatment administration of the agent into the epidermis is achieved by using an electrode current density which is 0.25 to 5 μA/cm², and an electrode voltage which is smaller than 1.75 V.

2. The method of claim 1, wherein the electrical potential difference is smaller than 1.0 V.

3. The method of claim 1, wherein the electrical potential difference is about 1.0 V to 1.75 V.

4. The method of claim 1, wherein the electrical potential difference and the current density are selected such that the skin will exhibit said second polarization state for 0.1 to 300 s after removing the device and electricity applied on the skin.

5. The method according claim 1, wherein said part of the individual's skin has an area of at least 1 mm².

6. The method according to claim 1, wherein at least one of the electrodes is applied on said part of the skin.

7. The method according to claim 1, wherein said part of the skin is subjected to treatment while it is in said second polarization state.

8. The method according to claim 1, wherein the step of subjecting said part of the skin to further treatment comprises applying an active agent on said part of the skin while it is in said second polarization state.

9. The method according to claim 1, wherein an active agent is applied to said part of the skin, either simultaneously with or subsequent to the supply of the current to the skin.

10. The method according claim 1, wherein a non-medical agent is applied on the skin while it is in the second polarization state.

11. The method according to claim 1, wherein said part of said skin is healthy, non-wounded skin.

12. The method according to claim 10, wherein said agent is supplied in uncharged form or in charged form, or wherein the agent is embedded in a charged or polar composition.

13. The method according to claim 10, wherein the agent is supplied as a topical formulation.

14. The method according to claim 1, wherein the depth into the skin to which the active agent penetrates, is further adjusted:
by selecting one or more of the following: appropriate properties of the formulation; viscosity; conductivity; adhesiveness; concentration of the buffer; concentration of the electrolyte and the concentration of the agent in the composition;
by selecting an appropriate period of time that the treatment process is allowed to proceed;
by selecting the magnitude of the voltage;
by selecting the magnitude of the current; or by a combination thereof

15. The method according to claim 1, wherein a wearable current source is used.

16. The method according to claim 15, wherein the wearable current source is selected from the group of a battery with stored electrical energy, a fuel cell, and a half enzymatic or Zn air battery.

17. The method according to claim 1, wherein a positively charged active agent, a positively charged liposome or the like including the active agent is administered to the individual by placing a formulation comprising the a active agent in contact with the surface of the positive electrode before pressing this electrode against the individual's skin.

18. A device in the form of a patch for delivering of an active cosmetic or medical agent in neutral form or in charged form to an individual's skin, said device comprising:
a delivery unit comprising a pair of electrodes including a positive electrode and a negative electrode, both to be contacted with the individual's skin, wherein an electrical current in the skin can be generated to form a potential difference between said electrodes, and
an electrical power source comprising a biofuel cell or a cascade of biofuel cells which comprise a catalyst and which can be activated with an aqueous salt solution for providing a current and voltage to said electrodes, said power source comprising an anode and a cathode and an electrically conductive component between said anode and cathode,
said delivery unit and said power source being included in the patch,
wherein the device is configured to polarize the skin and administrate the agent into the epidermis with an electrode current density which is 0.25 to 5 μA/cm², and an electrode voltage which is smaller than 1.75 V.

19. The device according to claim 18, wherein one of the electrodes is capable of being contacted with the skin via a layer of active substance.

20. The device according to claim 18, wherein the power source further comprises:
a cathode comprising a dry conductive layer containing an enzyme, wherein said
cathode catalyses reduction of ambient oxygen to water by uptake of electrons and hydrogen ions, and
an anode comprising a dry conductive layer containing an enzyme capable of oxidising or dehydrogenating a carbohydrate, and a fuel layer comprising essentially dry carbohydrate, wherein said anode catalyses oxidation of the carbohydrate, thereby releasing electrons and hydrogen ions.

21. The device according to claim 18, further comprising a positively charged active agent which is capable of being administered to the individual by placing a formulation comprising the active substance in contact with the surface of the positive electrode before pressing this electrode against the individual's skin.
